(19) United States
(12) Patent Application Publication
(10) Pub. No.: US 2005/0069898 A1
(43) Pub. Date: Mar. 31, 2005

(54) Lyophilized beads containing mannitol

(75) Inventors: Byung Sook Moon, Palo Alto, CA (US); Martin Jones, Walnut Creek, CA (US); Johnny Valdez, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cepheid, Sunnyvale, CA (US)

(21) Appl. No.: 10/672,266

(22) Filed: Sep. 25, 2003

(51) Int. Cl. C12Q 1/68; C12P 19/34; C12N 11/04

(52) U.S. Cl. 435/6; 435/91.2; 435/182

(57) ABSTRACT

Mannitol in certain weight percentages can be used to produce lyophilized beads of consistent size, consistent morphology, and reduced moisture content. These mannitol-containing lyophilized beads are useful in a variety of biological applications where precise reagent amounts are required or moisture-sensitive components are utilized. PCR technologies represent one biological application where mannitol-containing lyophilized beads can be used.

### FIELD OF THE INVENTION

This invention relates to reagent-containing lyophilized beads for use in biological reactions. In particular, it relates to compositions containing mannitol.

### BACKGROUND OF THE INVENTION

For years, scientists have encapsulated biological reagents in lyophilized beads. These lyophilized beads are produced for a variety of reasons. One is to increase efficiency and reduce experimental error in biological reactions. For example, in certain experiments, reaction components must be mixed in a step-wise fashion or simultaneously at the outset. Adding and mixing trace amounts of each component in a separate manner for every test sample results in frequent experimental errors. Especially when numerous samples are to be analyzed in a short period of time, the inefficiency and accompanying errors represent a serious obstacle to the success of the experiments. Lyophilized beads with premeasured reaction components represent one way of solving this problem.

Lyophilized beads are also used to reduce contact of the biological reagents with water. This hygroscopic reduction can further reduce experimental measuring errors. For example, if the bead material readily absorbs water from the environment, the weight and morphology of the beads will vary over time, thus introducing additional error into the experiment. Producing lyophilized beads formed from less hygroscopic materials addresses this concern.

Hygroscopic reduction in lyophilized beads also has the benefit of increasing the stability of the biological reagents encapsulated in the beads. For example, moisture-sensitive substances may degrade as the amount of water in the bead increases. Beads formed from low hygroscopic materials can therefore also show increased reaction component activity over high hygroscopic beads. This has led to the use of low hygroscopic beads as cyroprotectant agents. Further information on these cryoprotectant properties can be found in Maa, et al., Curr. Pharm. Biotechnol. 3:283-302 (2000); Prestrelski, et al., Arch. Biochem. Biophys. 303(2):465-73 (1993).

New lyophilized bead materials could aid scientists in their utilization of a variety of biological reactions. Polymerase chain reaction technology (hereinafter referred to as “PCR”) would certainly benefit from these new beads. PCR allows a nucleic acid sequence of interest to be amplified by more than a millionfold, provided that at least part of its nucleotide sequence is already known. Since trace amounts of a variety of materials, i.e., primers, reaction buffer, MgCl₂, KCl, dNTPs (deoxynucleoside triphosphates) (dATP, dCTP, dGTP and dTTP) and a polymerase or ligase, are required for a PCR reaction mixture, a premixed bead would aid efficiency and reduce experimental error. In addition, a bead with low hygroscopy would protect the enzymes, enzyme substrates, and dNTPs, that degrade as bead moisture increases. Accordingly, there is a need for a lyophilized bead with low hygroscopic properties in PCR. This invention solves this problem and other concerns as well.

### SUMMARY OF THE INVENTION

The present invention provides lyophilized beads containing mannitol in certain weight percentages. These mannitol-containing lyophilized beads are useful in a variety of biological applications where precise reagent amounts are required or moisture-sensitive components are utilized, such as PCR.

Thus, in a first aspect, the invention provides a lyophilized bead suitable for use in the amplification of a nucleic acid sequence. This lyophilized bead comprises a thermally stable enzyme and mannitol. The amount of mannitol in the lyophilized bead, on a weight of mannitol/weight of bead percentage, is between about 53% and about 75%. In one embodiment, the amplification of the nucleic acid sequence occurs in a reaction mixture having a volume of between about 5 μL and about 200 μL. In another embodiment, the lyophilized bead also contains a nucleoside triphosphate or a derivative thereof. In still another embodiment, the lyophilized bead has an average cross-section of between about 1 millimeter and about 4.5 millimeters. In yet another embodiment, lyophilized bead contains a nucleoside triphosphate or a derivative thereof. In another embodiment, the weight percentage of the lyophilized bead is between about 62% and about 75% (w/w). In another embodiment, the weight percentage of the lyophilized bead is between about 68% and about 75% (w/w). In still another embodiment, the thermally stable enzyme is selected from the group consisting of polymerase, ligase, and combinations thereof. In yet another embodiment, the lyophilized bead also contains a hot start methodology. In still another embodiment, the lyophilized bead also contains HEPES. In still another embodiment, the lyophilized bead also contains a reverse transcriptase. In another embodiment, the lyophilized bead also contains an internal control. In another embodiment, the lyophilized bead also contains a probe.

In a second aspect, the invention provides a lyophilized bead suitable for use in the amplification of a nucleic acid sequence. This lyophilized bead comprises a forward polynucleotide primer, a reverse polynucleotide primer, and mannitol. This lyophilized bead also has a weight percentage of mannitol of between about 53% and about 75% (w/w). In one embodiment, the amplification occurs in a volume of between about 5 μL and about 200 μL. In another embodiment, the lyophilized bead has an average cross-section of between about 1 millimeter and about 4.5 millimeters. In yet another embodiment, the lyophilized bead has a weight percentage of mannitol of between about 62% and about 75% (w/w). In still another embodiment, the lyophilized bead has a weight percentage of mannitol of between about 68% and about 75% (w/w). In still another embodiment, the lyophilized bead also contains HEPES. In still further embodiments, the lyophilized bead contains an internal control. In another embodiment, the lyophilized bead also contains a probe. In some embodiments, the nucleic acid sequence being amplified is a bacterial, fungal, or viral nucleic acid sequence. In other embodiments, the bacterial nucleic acid sequence can be derived from Bacillus Anthracis, Yersinia pestis, Clostridium botulinum, Francisella tularensis, Group B Streptococcus, Neisseria gonorrhoeae, Chlamydia trachomatis, or Xylella fastidiosa. In still other embodiments, the viral nucleic acid sequences can be derived from Vaccinia, West Nile Fever virus, Equine Encephalitis virus, or Foot and Mouth Disease virus.

In a third aspect, the present invention provides a method for the amplification of a nucleic acid sequence. This method has two parts. In part (a), a lyophilized bead is dissolved in a liquid, thus forming a reaction mixture. The lyophilized bead comprises a thermally stable enzyme and mannitol. The amount of mannitol in the lyophilized bead, on a weight of mannitol/weight of bead percentage, is between about 53% and about 75%. In part (b), the reaction mixture is subjected to an amplification reaction. In one embodiment, the reaction mixture has a volume of between about 5 μL and about 200 μL. In another embodiment, the reaction mixture further comprises a nucleoside triphosphate or a derivative thereof. In yet another embodiment, the thermally stable enzyme is selected from the group consisting of polymerase, ligase, and combinations thereof. In some embodiments, the reaction mixture also contains a forward polynucleotide primer. In some embodiments, the reaction mixture also contains a reverse polynucleotide primer. In some embodiments, the reaction mixture also contains a probe. In some embodiments, the reaction mixture also contains a nucleic acid comprising the nucleic acid sequence. In some embodiments, the reaction mixture also contains HEPES. In some embodiments, the reaction mixture also contains an internal control. In some embodiments, the reaction mixture also contains a hot start methodology. In yet another embodiment, the reaction mixture has an average cross-section of between about 1 millimeter and about 4.5 millimeters.

In a fourth aspect, the present invention provides a method for the amplification of a nucleic acid sequence. This method has two parts. In part (a), a lyophilized bead is dissolved in a liquid, thus forming a reaction mixture. The lyophilized bead comprises a forward polynucleotide primer, a reverse polynucleotide primer, and mannitol. The lyophilized bead in this method has a weight percentage of mannitol of between about 53% and about 75% (w/w). In part (b), the reaction mixture is subjected to an amplification reaction. In one embodiment, the reaction mixture has a volume of between about 5 μL and about 200 μL. In some embodiments, the reaction mixture also contains a nucleoside triphosphate or a derivative thereof. In some embodiments, the reaction mixture also contains a probe. In some embodiments, the reaction mixture also contains a nucleic acid comprising the nucleic acid sequence. In some embodiments, the reaction mixture also contains HEPES. In some embodiments, the reaction mixture also contains a thermally stable enzyme. In some embodiments, the reaction mixture also contains an internal control. In yet another embodiment, the reaction mixture has an average cross-section of between about 1 millimeter and about 4.5 millimeters.

In a fifth aspect, the present invention provides a lyophilized bead suitable for use in the amplification of a nucleic acid sequence. This bead is prepared by a three part process. In part (a), an aqueous solution is created. In this aqueous solution is a thermally stable enzyme and mannitol. The aqueous solution has a concentration of mannitol of between about 0.38 M (moles of mannitol/liter of solution) and about 0.99 M (moles of mannitol/liter of solution). In part (b), the product of part (a) is quick-frozen. In step (c), the product of step (b) is freeze-dried. In some embodiments, the product of (c) has an average cross-section of between about 1 millimeter and about 4.5 millimeters. In some embodiments, the product of (c) also contains a nucleoside triphosphate or a derivative thereof. In some embodiments, the thermally stable enzyme is either a polymerase, a ligase, or a combination thereof. In some embodiments, the product of (c) also contains a reverse transcriptase. In some embodiments, the product of (c) also contains a hot start methodology. In some embodiments, the product of (c) also contains HEPES. In some embodiments, the product of (c) also contains a probe. In still other embodiments, the product of (c) also contains an internal control.

In a sixth aspect, the present invention provides a lyophilized bead suitable for use in the amplification of a nucleic acid sequence. This bead is prepared by a three part process. In part (a), an aqueous solution is created. In this aqueous solution is a forward polynucleotide primer, a reverse polynucleotide primer, and mannitol. The aqueous solution has a concentration of mannitol of between about 0.38 M (moles of mannitol/liter of solution) and about 0.99 M (moles of mannitol/liter of solution). In part (b), the product of part (a) is quick-frozen. In step (c), the product of step (b) is freeze-dried. In some embodiments, the product of (c) has an average cross-section of between about 1 millimeter and about 4.5 millimeters. In some embodiments, the product of (c) also contains a nucleoside triphosphate or a derivative thereof. In some embodiments, the product of (c) also contains a thermally stable enzyme. In some embodiments, the product of (c) also contains a reverse transcriptase. In some embodiments, the product of (c) also contains a hot start methodology. In some embodiments, the product of (c) also contains HEPES. In some embodiments, the product of (c) also contains a probe. In still other embodiments, the product of (c) also contains an internal control.

In a seventh aspect, the present invention provides a lyophilized bead suitable for use in microanalytic systems. The lyophilized bead contains a moisture-sensitive reactant and mannitol. The lyophilized bead has a weight percentage of mannitol of between about 53% and about 75% (w/w). The lyophilized bead also has an average cross-section of between about 1 millimeter and about 4.5 millimeters. In some embodiments, the weight percentage of mannitol in the lyophilized bead is between about 62% and about 75% (w/w). In other embodiments, the weight percentage of mannitol in the lyophilized bead is between about 68% and about 75% (w/w).

In an eighth aspect, the present invention provides a method of using a lyophilized bead in a microanalytic system. The lyophilized bead contains a moisture-sensitive reactant and mannitol. The lyophilized bead has a weight percentage of mannitol of between about 53% and about 75% (w/w). The lyophilized bead also has an average cross-section of between about 1 millimeter and about 4.5 millimeters. In some embodiments, the weight percentage of mannitol in the lyophilized bead is between about 62% and about 75% (w/w). In other embodiments, the weight percentage of mannitol in the lyophilized bead is between about 68% and about 75% (w/w).

### BRIEF DESCRIPTION OF THE DRAWINGS

FIG. 1: Pictures of the lyophilized beads of the invention with different excipient formulations. Picture A shows compositions containing 9.0% (w/v) trehalose. Picture B shows compositions containing 18.8% (w/v) trehalose. Picture C shows compositions containing 4.5% (w/v) mannitol. Picture D shows compositions containing 6.0% (w/v) mannitol. Picture E shows compositions containing 9.0% (w/v) mannitol. Picture F shows compositions containing 11.0% (w/v) mannitol.

### Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, analytical chemistry, and nucleic acid chemistry and hybridization described below are those well known and commonly employed in the art. The techniques and procedures are generally performed according to conventional methods in the art and various general references (see generally, Kochanowski, et. al., eds. Quantitative PCR Protocols (Methods in Molecular Medicine, Vol 26), Humana Press:Totowa, N.J., (1999), which is incorporated herein by reference), which are provided throughout this document. Standard techniques, or modifications thereof, are used for chemical syntheses and chemical analyses.

An “amplification reaction” or “the amplification of a nucleic acid sequence”, refers to any chemical, including enzymatic, reaction that results in increased copies of a nucleic acid sequence. Amplification reactions include polymerase chain reaction (PCR) and ligase chain reaction (LCR) (see U.S. Pat. Nos. 4,683,195 and 4,683,202; Innis, et al., eds, PCR Protocols: A Guide to Methods and Applications (1990)), strand displacement amplification (SDA) (Walker, et al., Nucleic Acids Res. 20(7):1691-1696 (1992); Walker, PCR Methods Appl. 3(1):1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350:91-92 (1991)), loop-mediated isothermal amplification (LAMP) (Notomi, et al., Nucleic Acids Res. 28:12 (2000)) and single primer amplification (SPA) (see U.S. Pat. Nos. 5,508,178, 5,595,891, and 5,612,199), rolling circle amplification (RCA) (Lisby, Mol. Biotechnol. 12(1):75-99 (1999)); Hatch, et al., Genet. Anal. 15(2):35-40 (1999)) and branched DNA signal amplification (bDNA) (see Iqbal, et al., Mol. Cell Probes 13(4):315-320 (1999)). Other amplification methods known to those of skill in the art include CPR (Cycling Probe Reaction), SSR (Self-Sustained Sequence Replication), QBR (Q-Beta Replicase), Re-AMP (formerly RAMP), RCR (Repair Chain Reaction), TAS (Transcription Based Amplification System), RT-PCR (Real Time PCR), and Reverse Transcriptase PCR.

A “bead”, as used herein, refers to a small, often round piece of material. A bead can have a spherical as well as a nearly spherical, e.g., elliptical, shape. In an exemplary embodiment, the beads have cross-sections which are between one millimeter and twenty-five millimeters. In another exemplary embodiment, the beads have cross-sections which are between five millimeters and fifteen millimeters. In yet another exemplary embodiment, the beads have cross-sections which are between one millimeter and six millimeters. In still another exemplary embodiment, the beads have cross-sections which are between one millimeter and four and a half millimeters.

A “microfluidic device,” as used herein, refers to a device having one or more fluid passages, chambers or conduits which have at least one internal cross-sectional dimension, e.g., depth, width, length, cross-section, etc., that is less than 1500 μm, and sometimes less than about 1000 μm, or about 500 μm, and typically between about 0.1 μm and about 500 μm.

A “microanalytic device,” as used herein, refers to a system wherein the analysis takes place in a volume of less than 250 μL.

“Nucleic acid” or “polynucleotide” or “nucleic acid sequences” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). The term nucleic acid sequences encompasses sequences which are obtained or purified from natural sources, as well as sequences which are obtained or constructed from recombinant or synthetic chemical processes.

A “probe” refers to a molecule that allows for the detecting of the polynucleotide sequence of interest. In certain embodiments, a probe comprises a polynucleotide sequence capable of hybridization to a polynucleotide sequence of interest. In other embodiments, a probe comprises an agent capable of intercalating into a polynucleotide sequence of interest. Examples of intercalating agents include ethidium bromide or SYBR Green. In other embodiments, the probe comprises a label. The probes are typically labeled either directly, as with isotopes, chromophores, lumiphores, chromogens, or indirectly, such as with biotin, to which a streptavidin complex may later bind. Thus, the labels of the present invention can be primary labels (where the label comprises an element that is detected directly or that produces a directly detectable element) or secondary labels (where the detected label binds to a primary label, e.g., as is common in immunological labeling). In some embodiments, labeled nucleic acid probes are used to detect hybridization. Nucleic acid probes may be labeled by any one of several methods typically used to detect the presence of hybridized polynucleotides. In some embodiments, label detection occurs through the use of autoradiography with ³H, ¹²⁵I, ³⁵S, ¹⁴C, or ³²P-labeled probes or the like. Other labels include, e.g., ligands which bind to labeled antibodies, fluorophores, chemiluminescent agents, intercalating agentsenzymes, and antibodies which can serve as specific binding pair members for a labeled ligand. An introduction to labels, labeling procedures, and detection of labels is found in Polak and Van Noorden Introduction to Immunocytochemistry, 2nd ed., Springer Verlag, N.Y. (1997); and in Haugland Handbook of Fluorescent Probes and Research Chemicals, a combined handbook and catalogue Published by Molecular Probes, Inc. (1996).

“Internal control,” as used herein, refers to a control reaction run in parallel, in the same container, and under the same conditions as a reaction of interest, that functions as a standard of comparison that is able to account for and sometimes adjust for extraneous influences on the reaction of interest.

A “moisture-sensitive reactant”, as used herein, refers to a component of the lyophilized bead that experiences degradation or a reduction in activity upon exposure to water. Examples of moisture-sensitive reactants include enzymes, enzyme substrates, and dNTPs.

An “amplification reagent” or “reagent for polynucleotide amplification”, as used herein, refers to a reagent for use to amplify nucleic acids in an amplification reaction. The reagent can, but need not, comprise all of the components required for an amplification reaction. Examples of components of an amplification reaction can include, but are not limited to: nucleic acids, including templates, primers or deoxynucleotide triphosphates, a DNA polymerase (e.g., Taq polymerase, polymerase complexed with a hot start antibody such as Platinum polymerase), buffers (e.g., Tris(2-Amino-2-hydroxymethyl-1,3-propanediol), HEPES (N-(2-Hydroxyethyl)piperazine-N′-(2-ethanesulfonic acid)), etc.), salts such as magnesium and/or potassium-based salts, disaccharides or disaccharide derivatives, carrier proteins, detergents, DMSO, or other like agents.

A “hot start methodology” refers to the sequestering of any component which is critical for the performance of a PCR reaction. For example, a polymerase can be inactivated through reversible binding with an antibody until a stringent primer-annealing temperature is reached. In another example, magnesium, a required component for polymerase activity, is maintained in a wax or oil barrier, thus inactivating the polymerase until the magnesium is dislodged from the barrier. Examples of different hot start methodologies are provided in the following articles (Chou, et al., Nucleic Acids Research 20: 1717-1723 (1992); Bassam, et al., Bio Techniques 14: 31-33 (1993); Horton, et al., Bio Techniques 16: 42-43 (1994); Kellogg, D. E., et al., Bio techniques 16: 1134-1137 (1994); Birch D. E., et al., Nature 381: 445-446 (1996); Bost, D. A., et al., The FASEB Journal 11: A1370 (1997)), which are incorporated herein by reference.

A “thermally stable enzyme”, as used herein, refers to a protein that is capable of catalyzing a reaction at an elevated temperature. Examples of thermally stable nucleic acid enzymes include Taq polymerases and ligases.

A “forward polynucleotide primer”, or 5′ primer, refers to a nucleic acid segment that is complementary to a coding nucleic acid sequence which is subject to amplification via PCR. This primer is used to initiate replication in PCR.

A “reverse polynucleotide primer”, or 3′ primer, refers to a segment of nucleic acids that is complementary to a non-coding nucleic acid sequence which is subject to amplification in PCR. This primer is used to initiate replication in PCR.

A “target” or “target nucleic acid” refers to a single or double stranded polynucleotide sequence sought to be amplified in an amplification reaction.

A “template” refers to a double or single stranded polynucleotide sequence that comprises the polynucleotide to be amplified, flanked by primer hybridization sites.

The symbol “w/w” refers to the dry weight of the excipient divided by the dry weight of the lyophilized bead.

The symbol “w/v” refers to the dry weight (in grams) of the excipient divided by the volume (in 100 mL) of the bead buffer formulation.

### DETAILED DESCRIPTION OF THE INVENTION

I. Introduction

The present invention demonstrates for the first time that mannitol in certain weight percentages can be used to produce lyophilized beads of consistent size, consistent morphology, and reduced moisture content. Beads that are formed using high-hygroscopic excipients, such as trehalose, suffer from a variety of problems. First, use of high hygroscopic excipients result in beads of inconsistent weight, due to variable amounts of water in the beads. Bead fusion is also a concern. Finally, excess water in high hygroscopic beads degrades both moisture sensitive biological reagents and experimental performance. Using mannitol in a lyophilized bead reduces hygroscopy to an acceptable level, thereby allowing for increased accuracy and increased protection of the bead's components. These mannitol-containing lyophilized beads are useful in a variety of biological applications where precise reagent amounts are required or moisture-sensitive components are utilized. PCR technologies represent one biological application where mannitol-containing lyophilized beads are used.

II. Lyophilized Beads

A “lyophilized bead” comprises an excipient and a biological reagent. The beads are produced by forming a bead buffer formulation (containing the excipient and biological reagent), creating the beads from the bead buffer formulation, and finally freeze-drying the beads. The produced bead can possess a variety of morphologies and shapes. Exemplary shapes include spherical, near spherical, elliptical or round structures. Exemplary morphologies include smooth or slightly roughened surfaces.

A. Excipient

Excipients are more or less inert substances added to a material in order to confer a suitable consistency or form to the material. A large number of excipients are known to those of skill in the art and can comprise a number of different chemical structures. Examples of excipients, which may be used in the present invention, include carbohydrates, such as sucrose, glucose, trehalose, melezitose, dextran, and mannitol; proteins such as BSA, gelatin, and collagen; and polymers such as PEG and polyvinyl pyrrolidone (PVP). The total amount of excipient in the lyophilized bead may comprise either single or multiple compounds.

In the present invention, the type of excipient is a factor in controlling the amount of bead hygroscopy. Lowering bead hygroscopy can enhance the bead's integrity (accuracy of weighing beads) and cryoprotectant abilities. However, removing all water from the bead would have deleterious effects on those reaction components, proteins for example, that require certain amounts of bound water in order to maintain proper conformations. In general, the excipient level in the beads should be adjusted to allow moisture levels of less than 3%. In some embodiments, the excipient is trehalose, mannitol, dextran, or combinations thereof.

The amount of excipient is also a factor in controlling the amount of bead hygroscopy. There are limits to the amount of excipient which can be added to form a bead. If the amount of excipient is too low, the material does not coalesce to form a bead-like shape. At the high end, excipient amounts are limited by the solubility of the excipient in the bead buffer formulation. The amount is also dependent upon the properties of the excipient. In an exemplary embodiment, trehalose is present from between 5% to 20% (w/v). In another exemplary embodiment, mannitol is present from between 2% to 20% (w/v). In yet another exemplary embodiment, mannitol is present from between 2% to 20% (w/v) and dextran is present from between 0.5% to 5% (w/v). In still another exemplary embodiment, mannitol is present in the lyophilized bead in a weight percentage of between 40% to 75% (w/w).

B. Biological Reagent

The present invention provides various biological reagents suitable for storage or use in biological reactions. In certain embodiments, the present invention can be used in amplification reactions and microfluidic devices.

i. Amplification Reactions

Amplification of a RNA or DNA template using reaction mixtures is well known (see U.S. Pat. Nos. 4,683,195 and 4,683,202; Innis et al., eds, PCR Protocols: A Guide to Methods and Applications (1990)). Methods such as polymerase chain reaction (PCR) and ligase chain reaction (LCR) can be used to amplify nucleic acid sequences of target DNA sequences directly, e.g., from mRNA, from cDNA, from genomic libraries or cDNA libraries as well as from organisms, environmental samples, or any other source of nucleic acids. The reaction can be carried out in a thermal cycler to facilitate incubation times at desired temperatures. PCR can also be used to detect the presence of a virus or bacteria, such as Bacillus Anthracis, in a cell sample (see Example 3).

Exemplary PCR reaction conditions typically comprise either two or three step cycles. Two step cycles have a denaturation step followed by a hybridization/elongation step. Three step cycles comprise a denaturation step followed by a hybridization step followed by a separate elongation step.

ii. Biological Reagents used as Amplification Reaction Components

Lyophilized beads can, but need not, have all components required to complete an amplification reaction. For example, in some circumstances it is convenient to store a mixture of some, but not all, of the components required for an amplification reaction. In some cases, all components but the nucleic acids are in the lyophilized bead. In some embodiments, only the components that are stable at room temperature or in a lyophilized mixture are included. In some aspects, the lyophilized beads are used in a microfluidic device. Exemplary microfluidic devices that employ amplification reaction mixtures include, e.g., the SmartCycler®, GeneXpert® and I-CORE® devices (Cepheid, Sunnyvale, Calif.). However, the advantages of the invention extend to all microfluidic devices, whether for use in amplification reactions or not.

Thus, in some aspects, the amplification mixture comprises a buffer, excipient, a carrier protein, magnesium, and an antifoam agent. Lyophilized beads that contain these mixtures are known as “Blank Beads”. In some aspects, the reaction mixture also comprises a polymerase such as a DNA polymerase (Taq polymerase, polymerases complexed with hot start antibodies such as Platinum polymerases (Invitrogen, Carlsbad, Calif.)), RNA polymerase, reverse transcriptase, and/or deoxynucleoside triphosphates (e.g., dATP, dCTP, dTTP, dGTP). Lyophilized beads that contain these mixtures are known as “Generic Beads”. In some embodiments, the amplification mixtures will comprise primers which correspond a particular DNA sequence of interest, as well as probes which will detect the presence of primer hybridization with the DNA sequence of interest. Lyophilized beads that contain these mixtures are known as “Assay Specific Beads”. Examples of bead buffer formulations of the invention are found in Example 1.

The bead buffer formulations of the invention can comprise any or all of the aforementioned or following amplification reaction mixture components. Those of skill in the art will recognize that a number of amplification reagents have been described in the art. The list below is not comprehensive.

Oligonucleotide Primers

The oligonucleotides that are used in the present invention as well as oligonucleotides designed to detect amplification products can be chemically synthesized. These oligonucleotides can be labeled with radioisotopes, chemiluminescent moieties, or fluorescent moieties. Such labels are useful for the characterization and detection of amplification products using the methods and compositions of the present invention.

The primer components may be present in the PCR reaction mixture at a concentration of, e.g., between 0.1 and 1.0 μM. The primer length can be between, e.g., 8-100 nucleotides in length. In order to aid in hybridization with the nucleic acid sequence, the primers in some embodiments have 50-60% G and C composition. In the choice of primer, it is preferable to have exactly matching bases at the 3′ end of the primer but this requirement decreases to relative insignificance at the 5′ end. In some embodiments, the primers of the invention all have approximately the same melting temperature.

Buffer

Exemplary buffers that may be employed, include, e.g., HEPES, borate, phosphate, carbonate, barbital, Tris, etc. -based buffers. See Rose et al., U.S. Pat. No. 5,508,178. The pH of the reaction should be maintained in the range of about 4.5 to about 9.5. See U.S. Pat. No. 5,508,178. The standard buffer used in amplification reactions is a Tris based buffer between 10 and 50 mM with a pH of around 8.3 to 8.8. See Innis et al., supra.

One of skill in the art will recognize that buffer conditions should be designed to allow for the function of all reactions of interest. Thus, buffer conditions can be designed to support the amplification reaction as well as any enzymatic reactions associated with producing signals from probes. A particular reaction buffer can be tested for its ability to support various reactions by testing the reactions both individually and in combination.

Salt Concentration

The concentration of salt present in the reaction mixture can affect the ability of primers to anneal to the target nucleic acid. See Innis et al. Potassium chloride is typically added up to a concentration of about 50 mM or more to the reaction mixture to promote primer annealing. Sodium chloride can also be added to promote primer annealing. See Innis et al.

Magnesium Ion Concentration

The concentration of magnesium ion in the reaction can be critical to amplifying the desired sequence(s). See Innis et al. Primer annealing, strand denaturation, amplification specificity, primer-dimer formation, and enzyme activity are all examples of parameters that are affected by magnesium concentration. See Innis et al. Amplification reactions can contain, e.g., about a 0.5 to 2.5 mM magnesium concentration excess over the concentration of dNTPs. The presence of magnesium chelators in the reaction can affect the optimal magnesium concentration. A series of amplification reactions can be carried out over a range of magnesium concentrations to determine the optimal magnesium concentration. The optimal magnesium concentration can vary depending on the nature of the target nucleic acid(s) and the primers being used, among other parameters. A common source of magnesium ion is MgCl₂.

Carrier Proteins

Carrier proteins useful in the present invention include but are not limited to albumin (e.g., bovine serum albumin) and gelatin.

Deoxynucleoside Triphosphate Concentration

Deoxynucleoside triphosphates (dNTPs) are added to the reaction to a final concentration of about 20 μM to about 300 μM. Each of the four dNTPs (G, A, C, T) are generally present at equivalent concentrations. See Innis et al.

Nucleic Acid Polymerase

A variety of DNA dependent polymerases are commercially available that will function using the methods and compositions of the present invention. For example, Taq DNA Polymerase may be used to amplify target DNA sequences. The PCR assay may be carried out using as an enzyme component a source of thermostable DNA polymerase suitably comprising Taq DNA polymerase which may be the native enzyme purified from Thermus aquaticus and/or a genetically engineered form of the enzyme. Other commercially available polymerase enzymes include, e.g., Taq polymerases marketed by Promega or Pharmacia. Other examples of thermostable DNA polymerases that could be used in the invention include DNA polymerases obtained from, e.g., Thermus and Pyrococcus species. Concentration ranges of the polymerase may range from 1-5 units per reaction mixture. The reaction mixture is typically between 20 and 100 μL.

In some embodiments, a “hot start” methodology polymerase can be used to prevent extension of mispriming events as the temperature of a reaction initially increases. Hot starts are particularly useful in the context of multiplex PCR. Examples of hot start methodologies include heat labile adducts attached to a polymerase or ligase requiring a heat activation step (typically 95° C. for approximately 10-15 minutes) or an antibody associated with the polymerase or ligase to prevent activation.

Other Agents

Assorted other agents are sometimes added to the reaction to achieve the desired results. For example, DMSO can be added to the reaction, though it is reported to inhibit the activity of Taq DNA Polymerase. Nevertheless, DMSO has been recommended for the amplification of multiple target sequences in the same reaction. See Innis et al. Non-ionic detergents (e.g. Tween-20) can also be added to amplification reactions. See Innis et al. In addition, methylisothiazolinone (MIT) can be added to the reaction mixture.

C. Methods of Producing the Lyophilized Beads

The compounds of the invention can be produced through a three step process. In the first step, the bead buffer formulation is created by mixing the biological reagents with the excipients. An illustration of the components of this formulation is in Example 1. In the second step, the buffer formulation is converted into a bead. In the third step, the beads are lyophilized.

i. Buffer Creation

Each of the various bead buffer components are added, one at a time, into a container which contains a stir plate and gently rotating stir bar. Any container which does not react with the bead buffer components can be used. Various methods of mixing are also possible, including, but not limited to, a standard propeller, disperser, sonicator, roller mill, or shaker. Cooling the mixture while mixing may be required for temperature sensitive biologicals like enzymes.

ii. Bead Formation

The final volume per dose of the solution is often small, between 2 μL to 15 μL, to allow a working volume of 5 μL to 200 μL when the lyophilized bead is dissolved in a working solution. Using a volumetric or gravimetric dispensing system such as those made by FMI or IVEC has been shown to work well. A time/pressure method such as that used to dispense adhesives also works well.

The composition and shape of the drying surface for the dispensed emulsion is important as it determines the drop shape and the ease of release from the surface after drying. In some embodiments, the dispensed emulsion is placed upon glass, polystyrene, wax paper, Delrin, or a coated aluminum pan (coatings can be nickel, Teflon, titanium nitrite and combinations thereof).

Bead formation can also occur by dropping the dispensed emulsion onto a cryogenic liquid or onto a cryogenically cooled solid surface. Cryogenic is defined as a liquefied or solidified gas having a normal boiling or sublimation point below about −75° C.; in some cases, this point is below about −150° C. In an exemplary embodiment, the cryogenic material is nitrogen, Freon, or carbon dioxide. The frozen beads are recovered and then freeze dried to a moisture content of less than about 10%. In some cases, the moisture content is less than 3%.

iii. Bead Lyophilization

Vacuum drying, evaporation, and freeze-drying of the solution can be utilized for drying the bead material. A standard freeze-drier (such as a VirTis Advantage and/or GENESIS) with a control modified to allow operation at partial vacuums can be used. Additionally, a constant purge of dry nitrogen into the chamber can provide gas flow to carry moisture away from the bead material.

The problems to overcome in drying are loss of biological activity and loss of droplet shape. If the drying proceeds too quickly, biological activity is lost. Slowing the drying down by increasing the chamber pressure and lowering the purge gas rate produces increased biological activity of the dried droplets. Higher pressures also produce more consistently shaped drops. If the pressure is too low, the drops expand and produce an unacceptable range of shapes. The lyophilized beads of the invention are produced by keeping the chamber pressure at 100 mTorr for the drying step, which can take approximately one day.

The formed beads can also be dispensed onto a cryogenic surface and dried through a freeze-drying process. A typical successful freeze-drying profile is shown in Table 2.

The following examples are offered by way of illustration and not by way of limitation.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

General

In the examples below, unless otherwise stated, temperatures are given in degrees Celsius (° C.); operations were carried out at room or ambient temperature, “rt,” or “RT,” (typically a range of from about 18-25° C.); yields are provided for illustration only; and the following conventional abbreviations are also used: L (liter(s)), mL (milliliters), mmol (millimoles), g (grams), mg (milligrams), min (minutes), and h (hours). All chemicals were purchased from Sigma-Aldrich, unless otherwise noted.

### Example 1

Bead Production

The lyophilized beads of the invention were produced in a three step process: A) buffer creation; B) bead formation; and C) bead lyophilization.

A) Buffer Creation

HEPES sodium salt hydrate (2.16 g; MW=260.3), HEPES (0.405 g; MW=238.3), Excipient(s) (See Table 1 for amounts), KCl (0.448 g; MW=74.55), MgCl₂ (0.23 g; MW=95.21), Bovine Serum Albumin (BSA) (0.18 g), and 2-Methyl-4-IsoThiazolin-3-one (MIT) (0.10 g; MW=151.6) were individually added to a stirred solution of water, which was then brought to a final volume of 100 mL. Stirring was accomplished with a stir bar and stir plate. Each component was allowed to dissolve completely before the next component was added. The pH of this buffer system was measured to ensure it was at pH 8.00+/−0.1. The buffer system was next filtered using a sterile 0.2 μm Corning brand Vacuum Filter/Storage System. A clean stirring bar was added to the filtered lyophilization buffer solution, and then Tween 20 (2 mL of 10% soln, purchased from Pierce-DH57656) and AF SE-15 (0.24 mL of 10% soln) were slowly added. The lyophilization buffer solution was then mixed thoroughly for one hour and stored at 2-8° C. The solid weight of mannitol or trehalose added, % of excipients (w/v) in the pre-lyophilized buffer system, as well as the weight % mannitol or trehalose in the lyophilized bead are shown in Table 1.

TABLE 1

| | Solid weight | | |
|---|---|---|---|
| Mannitol % w/v | gm/100 mL | Mannitol % w/w | Dextran % w/v |
| 4.5 | 10.523 | 42.76 | 2.5 |
| 6.0 | 12.023 | 49.90 | 2.5 |
| 7.0 | 13.023 | 53.75 | 2.5 |
| 8.0 | 14.023 | 57.05 | 2.5 |
| 9.0 | 15.023 | 59.91 | 2.5 |
| 11.0 | 17.023 | 64.62 | 2.5 |

| Trehalose % w/v | Solid weight/100 mL | Trehalose % w/w |
|---|---|---|
| 18.80 | 22.323 | 84.22 |
| 9.0 | 12.523 | 71.87 |

B) Bead Formation

Bead formation was conducted using the IVEK Digispense™ 700 system (IVEK Corporation, North Springfield, Vt.) dispensing system. The system was loaded with the liquid bead buffer formulation, which was dispensed in 12.50 μL amounts into liquefied nitrogen contained in a Dewar flask with insert dividers. Dispensing the liquid bead buffer formulation into liquid nitrogen caused spontaneous bead formation. Bead freezing required submersion in the liquid nitrogen for approximately 20 to 30 seconds. During this period, the beads were maintained in separate areas by the insert dividers to prevent bead fusion.

At the end of the bead forming process the frozen beads were collected and transferred into a glass vial with a slotted rubber stopper inserted halfway into the vial (100-200 frozen beads per vial). The vials containing the frozen beads were transferred into the lyophilizer chamber which was pre-chilled to −70° C.

C. Bead Lyophilization

The lyophilization cycle parameters are shown in Table 2. A VirTis Advantage 2.0 EL lyophilizer (VirTis, Gardiner, New York) was used.

TABLE 2

| | Shelf | Condenser | Vacuum | Time |
|---|---|---|---|---|
| Lyophilization Stage | (° C.) | (° C.) | (mTorr) | (hours) |
| Pre-Freeze Shelf | −70° C. | −70° C. | Off | 1 |
| Load Product Additional | −50° C. | −70° C. | Off | 1 |
| Freeze | | | | |
| Vacuum Auto Initiated | −50° C. | −70° C. | 100 | 2 |
| Set Primary Dry Temp. | −35° C. | −70° C. | 100 | 16 |
| Set Secondary Dry Temp. | 20° C. | −70° C. | 100 | 6 |
| Post Heat | 20° C. | −70° C. | 100 | 1 |
| | | | Total Time | 27 |

After the product completed the lyophilization cycle, nitrogen was introduced into the lyophilization chamber, and the vacuum seal was released. The slotted and stoppered vials were pushed into glass vials and sealed to protect them from ambient conditions. The beads were then moved to a low humidity environment (Dry Box <10% relative humidity) and transferred into plastic 0.5 mL conical reaction tubes. The tubes were sealed, placed into pouches with desiccant, and the pouches were subsequently heat-sealed.

### Example 2

Characterization of the Lyophilized Beads

Several physical characteristics of the beads were measured, including: cross-section, shape, roughness, size uniformity, bead integrity, extent of crystallinity, structure, moisture content, and phase transition temperature.

i. Bead Cross-Section

Bead cross-section was measured using the See-Brez Video Microscope (Quality Control Solutions Inc., Temecula, Calif.). Three beads from each excipient formulation of Table 1 were measured. Each bead was measured twice, for a total of six measurements per excipient formulation. Raw and statistical data are shown in Table 3.

TABLE 3

| | Trehalose | | | | | | | |
|---|---|---|---|---|---|---|---|---|
| | % w/v | | Mannitol % w/v | | | | | |
| | 18.8 | 9.0 | 4.5 | 6.0 | 7.0 | 8.0 | 9.0 | 11.0 |
| Unit | (mm) | (mm) | (mm) | (mm) | (mm) | (mm) | (mm) | (mm) |
| Bead #1 | 2.2890 | 1.8260 | 2.1780 | 2.8190 | 2.8195 | 2.9320 | 2.8671 | 2.8700 |
| | 2.5430 | 1.7715 | 2.2020 | 2.8245 | 2.7595 | 2.8635 | 2.7145 | 2.8435 |
| Bead #2 | 2.4470 | 2.0160 | 2.1515 | 2.7605 | 2.6375 | 2.8010 | 2.8365 | 2.8865 |
| | 2.1675 | 2.0710 | 2.2170 | 2.7365 | 2.7095 | 2.7840 | 2.7149 | 2.8300 |
| Bead #3 | 2.1875 | 1.8212 | 2.2130 | 2.8835 | 2.6655 | 2.8590 | 2.8265 | 2.8570 |
| | 2.2450 | 1.7900 | 2.2315 | 2.7465 | 2.7500 | 2.8585 | 2.7700 | 2.8500 |
| Mean | 2.31 | 1.88 | 2.20 | 2.80 | 2.72 | 2.85 | 2.79 | 2.86 |
| SD | 0.15 | 0.13 | 0.03 | 0.06 | 0.07 | 0.05 | 0.07 | 0.02 |
| % CV | 6.50 | 6.77 | 1.33 | 2.04 | 2.44 | 1.84 | 2.33 | 0.70 |
| N | 6 | 6 | 6 | 6 | 6 | 6 | 6 | 6 |

ii. Bead Morphologies

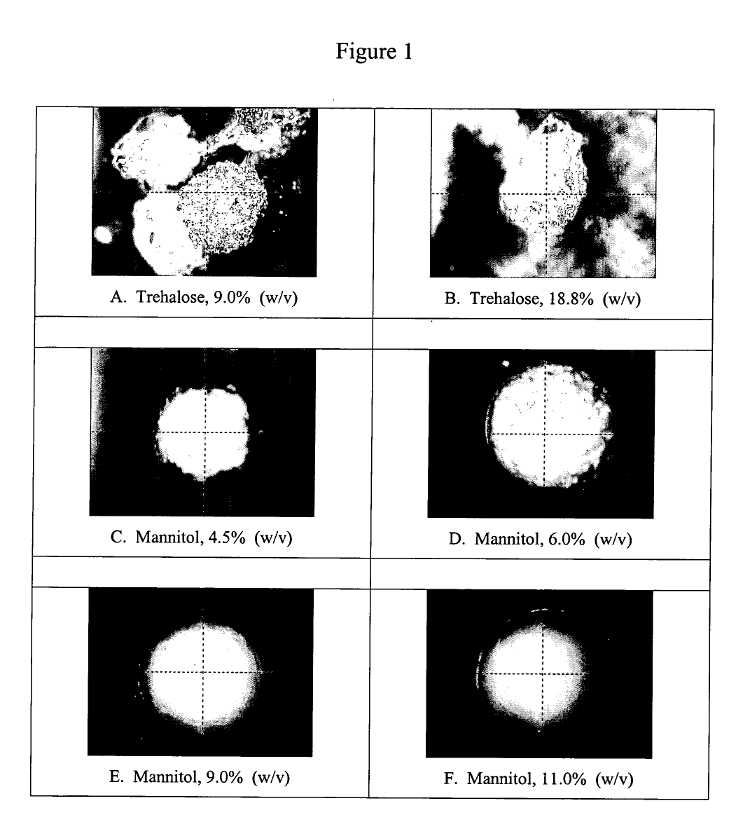

Bead shape, roughness, size uniformity, and bead integrity were observed with the unaided eye and with a See-Breez microscope. This data is shown in Table 4. Photographs taken of the beads with the Sea-Breez microscope are shown in FIG. 1.

TABLE 4

| | | | Size | Bead | |
|---|---|---|---|---|---|
| | Shape | Roughness | Uniformity | Integrity | Comments |
| Trehalose | | | | | |
| 18.8% w/v | − | + | − | + | The material did not lyophilize. The |
| [84.22% w/w] | | | | | product lost uniformity in shape. It |
| | | | | | formed a shiny, clear irregular shaped |
| | | | | | mass that adhered to the bottom of |
| | | | | | container (not transferable). The pre- |
| | | | | | lyophilization bead shape was |
| | | | | | completely lost. |
| 9.0% w/v | − | + | − | + | Similar to trehalose 18.8% results. |
| [71.87% w/w] | | | | | However, bead size was smaller. |
| Mannitol | | | | | |
| 4.5% w/v | − | + | − | $ | The materials were transferable. |
| [42.76% w/w] | | | | | Lyophilized material was semi- |
| | | | | | spherical with many sharp protrusions |
| | | | | | forming a rough surface. |
| 6.0% w/v | @ | ++/$ | + | $ | The materials were transferable, with |
| [49.90% w/w] | | | | | a well-defined spherical shape. |
| | | | | | However, many pits and craters |
| | | | | | existed on the surface. |
| 7.0% w/v | @ | +/$ | + | $ | Visual inspection with the naked eye |
| [53.75% w/w] | | | | | indicated a spherical shape with |
| | | | | | minor surface abnormalities. |
| 8.0% w/v | @ | $ | + | $ | Smooth spherical beads with |
| [57.05% w/w] | | | | | excellent morphology. |
| 9.0% w/v | @ | $ | + | $ | Smooth spherical beads with |
| [59.91% w/w] | | | | | excellent morphology. |
| 11.0% w/v | @ | $ | + | $ | Smooth spherical beads with |
| [64.62% w/w] | | | | | excellent morphology. |

Key:
Shape:
X = did not form beads
− = overall bead shape was deformed (neither spherical nor elliptical)
@ = elliptical or spherical bead shape was maintained
Roughness
+ = beads had a rough surface texture
$ = beads had a smooth surface texture
Size Uniformity
X = did not form beads
− = beads did not retain pre-lyophilized size
+ = beads retained pre-lyophilized size
Bead Integrity
X = did not form beads
− = large number of the beads were fused together
+ = small number of the beads were fused together
$ = beads were not fused together

iii. Extent of Crystallinity

Extent of crystallinity was assessed using powder x-ray diffraction (PXRD). One vial of each lot was sent to an outside contract laboratory to conduct powder x-ray diffraction (PXRD).

For PXRD, a copper x-ray source was operated at 40.0 kV, 35.0 mA and scans were made from 4.0 to 45.0 degrees (2θ). The unit cell size was resolved from the angular positions of the diffraction peaks and the arrangement of the atoms within the unit cell was associated with the relative intensities of the peaks. These angular positions and relative intensities allowed for comparisons with other known diffraction patterns. The International Center for Diffraction Data (ICDD) standard library was used as a reference in determining the presence of crystalline polymorphs (α, β, δ) of mannitol in the sample.

The x-ray diffractograms for the dried bead samples were compared against bead samples known to contain either 1) a glassy, amorphous structure or 2) a known crystalline diffraction pattern. Beads were prepared under nitrogen in a glove bag, covered with a polycarbonate film to protect them from moisture, and analyzed. The powder x-ray diffractogram for the glassy, amorphous structure demonstrated an amorphous halo with no evidence of crystallinity. The powder x-ray diffractogram for the lyophilized beads of the invention were consistent with the δ-polymorph of crystalline mannitol. Although this diffractogram shows high crystallinity, because the peaks do not return completely to the baseline it can be assumed that the lyophilized beads also contain some amount of amorphous material.

iv. Scanning Electron Microscopy

One vial of each lot was sent to an outside contract laboratory to conduct scanning electron microscopy. The SEM photomicrographs were conducted at 100X, 300X, 1000X, and 3000X magnifications of both the surface and a fracture of the beads.

Morphology was determined via scanning electron microscopy (SEM). Both samples were prepared for SEM under nitrogen in a glove bag. Surface photos of the glassy, amorphous material showed a smooth surface with small cracks throughout. The cross section displayed a thick (0.25 mm to 0.5 mm) skin which differed in morphology from the center of the bead. The cross section of the skin of this bead lacked ice crystal formation. The center of the bead revealed a lack of repeating shapes or structures and displayed a more webbed appearance. At high magnifications, the material was smooth and lacked any sharp corners.

The surface of the lyophilized beads of the invention were slightly porous with pore cross-sections of approximately 10 μm. At high magnifications, numerous repeating regular geometric shapes were seen protruding from the surface. The cross section of the lyophilized bead revealed a fairly consistent morphology across the bead. The cross section of the skin of this bead lacked ice crystal formation. At high magnifications, the material displayed a slightly uneven surface with what appeared to be outgrowths of irregular shapes protruding from the surface.

v. Moisture Content

Residual moisture determination was performed on a Mettler Toledo DL36 KF Coulometer attached to a GA42 Mettler Toledo printer and AG104 Mettler Toledo electronic balance. Moisture experiments were performed using five sets of 28 lyophilized blank beads containing 64.6% (w/w) mannitol and 19.9% (w/w) dextran. Five mL Wheaton serum clear glass vials, Lot Number 1192752-02 with rubber stoppers were used to perform the extraction. Vials and syringes were washed in methanol and over dried at 50° C. for at least 60 min. Bead filling into vials was performed in the glove box at a relative humidity of 5% at 23° C. and sealed with a rubber stopper. All weighing was done in duplicate and the average taken; however, the calculation of percentage of moisture was done individually for each injection, and then averaged. Water was extracted from the lyophilized beads using methanol. 1.5 mL methanol (Riedel-de Haen® 34741, Lot Number 1085A) was injected into the vial by piecing the rubber stopper with the syringe needle (2.5 mL Hamilton gastight syringe with a Mettler Part Number 71483 hypodermic needle Number 12/80). Throughout the extraction period vials were swirled gently to mix the contents at 15 minutes intervals. Injection into the KF titration reaction vessel was performed with a 1 mL Hamilton gastight syringe attached with a Mettler Part Number 71483 hypodermic needle Number 12/80. The syringe was washed with approximately 200 μL of extracted sample prior to injection. A Riedel-de Haen® #334828, Lot Number 11910 Hydranal Water Standard 1.00 was used to verify the KF performance at the beginning of the run. If the standard gave a result of within 10% of 0.1% moisture target content then the run was verified as acceptable. The % moisture for each of the five sets of tested lyophilized beads, as well as aggregate statistical information, is provided in Table 5.

TABLE 5

| Vial # | Blank | 1 | 2 | 3 | 4 | 5 |
|---|---|---|---|---|---|---|
| Sample Bead Weight (gm) | NA | 47.1 | 46.9 | 47.0 | 45.5 | 46.1 |
| Total Moisture Sample (mg) | NA | 0.9837 | 0.9784 | 0.9235 | 0.8863 | 1.0384 |
| | | 1.0522 | 0.9670 | 0.9188 | 0.8804 | 1.0267 |
| % Moisture Sample = Total | NA | 2.09 | 2.09 | 1.96 | 1.95 | 2.25 |
| Moisture (mg)/Sample Wt (mg) | | 2.23 | 2.06 | 1.95 | 1.93 | 2.23 |
| % Moisture Blank from printer | 0.0178 | NA | NA | NA | NA | NA |
| Corrected % Sample Moisture = | NA | 2.07 | 2.07 | 1.94 | 1.93 | 2.23 |
| (% Moisture Sample) − | | 2.21 | 2.04 | 1.93 | 1.91 | 2.21 |
| (% Moisture Blank) | | (2.14) | (2.06) | (1.94) | (1.92) | (2.22) |

Aggregate Statistics

| | |
|---|---|
| Mean (% Moisture) | 2.06 |
| SD | 0.13 |
| CV (%) | 6.24 |
| N | 5 |

vi. Phase Transition Temperatures

Phase transition temperatures were measured using high temperature differential scanning calorimetry (HT-DSC). Approximately 9 mg of material (5 beads) were placed in an aluminum sample pan with a lid crimped in place in a nitrogen filled glove box. During warming, evolution or uptake of heat for the sample reflected the differences in energy as the sample underwent a thermal event. This difference in heat energy was recorded for analysis of the results. The scan data was recorded and graphed using the Pyris 4.0 software.

Differential scanning calorimetry scans were run at 2° C. and 10° C. per minute. Table 6 below includes the primary thermal events evident in each of the samples. The lack of a notable thermal event on a particular scan is indicated by “none”.

TABLE 6
Glassy, amorphous material

| | Endo- | Endo- | Endo- |
|---|---|---|---|
| Rate | therm 1 | therm 2 | therm 3 |
| 2° C./min | 57.87 | 84.45 | 195.84 |
| 10° C./min | 57.99 | 83.85 | 192.22 |

Lyophilized Beads of the Invention

| | Endo- | Endo- | Endo- | Endo- | Endo- |
|---|---|---|---|---|---|
| Rate | therm 1 | therm 2 | therm 3 | therm 4 | therm 5 |
| 2° C./min | 61.17 | none | 117.19 | 137.32 | 148.0 |
| 10° C./min | 60.23 | 73.68 | 119.53 | 138.65 | 149.16 |

### Example 3

Use of the Lyophilized Beads in the Detection of Bacillus Anthracis using Real-Time Polymerase Chain Reaction (RT-PCR)

Bacillus Anthracis is the bacteria which causes the acute infectious disease anthrax in humans. A rapid RT-PCR assay utilizing the lyophilized beads of the invention was used for the detection of this bacteria.

Two types of lyophilized beads were added to the PCR vial in this assay. The first type was a generic bead (GB), which, in addition to the components specified in Example 1, contained a “Hotstart” Platinum DNA polymerase. Hotstart polymerases are precomplexed with specific monoclonal antibodies to render the polymerase inactive. The second bead type was an assay specific bead (ASB), which, in addition to the Example 1 components, also contained primers for Bacillus Anthracis and probes. One of each type of bead was placed in the PCR vial. A sample containing lysed Bacillus Anthracis bacteria was then added to the PCR vial, along with enough buffer to both dissolve the beads and bring the total reaction volume to 50 μL. In a separate experiment, purified Bacillus Anthracis DNA was used. The 50 μL solution was divided into two 25 μL solutions, dispensed into two tubes, and then introduced into a Cepheid Smart Cycler analyzer (Cepheid, Sunnyvale, Calif.). The solutions were then subjected to an initial heating to 95° C. for 30 seconds. Next, the solutions were subjected to 45 PCR cycles. The PCR cycle parameters were: 95° C. for 1 second and 65° C. for 20 seconds. The initial amount of Bacillus Anthracis DNA, the amount of fluorescence observed at the conclusion of 45 cycles (end point), mean end point, number of cycles required before fluorescence was viewed (cycle threshold), and mean cycle threshold are shown in Table 7.

TABLE 7

| Initial Amt of Bacillus | | Mean | | |
|---|---|---|---|---|
| Anthracis DNA | End | End | Cycle | Mean Cycle |
| (pg/reaction) | Pt (FU) | Pt (FU) | Threshold | Threshold |
| Neg | −0.9 | | 0 | |
| Neg | −2.2 | | 0 | |
| 0.01 | 102.9 | | 36.2 | |
| 0.01 | 177.5 | | 35.1 | |
| 0.01 | 143.0 | | 36.5 | |
| 0.01 | 287.3 | 177.7 | 35.2 | 35.7 |
| 0.1 | 361.1 | | 33.0 | |
| 0.1 | 386.5 | | 32.6 | |
| 0.1 | 334.0 | | 32.3 | |
| 0.1 | 361.4 | 360.8 | 31.9 | 32.4 |

1. A lyophilized bead suitable for use in the amplification of a nucleic acid sequence, said lyophilized bead comprising:
a thermally stable enzyme; and
mannitol;
wherein said lyophilized bead has a weight percentage of said mannitol of between about 53% and about 75% (w/w).

2. The lyophilized bead of claim 1, wherein said amplification occurs in a reaction mixture comprising a volume of between about 5 μL and about 200 μL.

3. The lyophilized bead of claim 1, further comprising a nucleoside triphosphate or a derivative thereof.

4. The lyophilized bead of claim 1, wherein said lyophilized bead has an average cross-section of between about 1 millimeter and about 4.5 millimeters.

5. The lyophilized bead of claim 1, wherein said weight percentage is between about 62% and about 75% (w/w).

6. The lyophilized bead of claim 5, wherein said weight percentage is between about 68% and about 75% (w/w).

7. The lyophilized bead of claim 1, wherein said thermally stable enzyme is selected from the group consisting of polymerase, ligase, and combinations thereof.

8. The lyophilized bead of claim 1, further comprising a hot start methodology.

9. The lyophilized bead of claim 1, further comprising HEPES.

10. The lyophilized bead of claim 1, further comprising a probe.

11. The lyophilized bead of claim 1, further comprising a reverse transcriptase.

12. The lyophilized bead of claim 1, further comprising an internal control.

13. A lyophilized bead suitable for use in the amplification of a nucleic acid sequence, said lyophilized bead comprising:
a forward polynucleotide primer;
a reverse polynucleotide primer; and
mannitol;
wherein said lyophilized bead has a weight percentage of said mannitol of between about 53% and about 75% (w/w).

14. The lyophilized bead of claim 13, wherein said amplification occurs in a reaction mixture comprising a volume of between about 5 μL and about 200 μL.

15. The lyophilized bead of claim 13, wherein said lyophilized bead has an average cross-section of between about 1 millimeter and about 4.5 millimeters.

16. The lyophilized bead of claim 13, wherein said weight percentage is between about 62% and about 75% (w/w).

17. The lyophilized bead of claim 16, wherein said weight percentage is between about 68% and about 75% (w/w).

18. The lyophilized bead of claim 13, further comprising HEPES.

19. The lyophilized bead of claim 13, further comprising a probe.

20. The lyophilized bead of claim 13, further comprising an internal control.

21. The lyophilized bead of claim 13, wherein said nucleic acid sequence is selected from the group consisting of bacterial, fungal, and viral nucleic acid sequences.

22. The lyophilized bead of claim 21, wherein said bacterial nucleic acid sequence is derived from a member selected from the group consisting of Bacillus Anthracis, Yersinia pestis, Clostridium botulinum, Francisella tularensis, Group B Streptococcus, Neisseria gonorrhoeae, Chlamydia trachomatis, and Xylella fastidiosa.

23. The lyophilized bead of claim 21, wherein said viral nucleic acid sequence is derived from a member selected from the group consisting of Vaccinia, West Nile Fever virus, Equine Encephalitis virus, and Foot and Mouth Disease virus.

24. A method for the amplification of a nucleic acid sequence, said method comprising:
(a) dissolving a lyophilized bead in a liquid, wherein said lyophilized bead comprises:
a thermally stable enzyme; and
mannitol;
wherein said lyophilized bead has a weight percentage of said mannitol of between about 53% and about 75% (w/w), thus forming a reaction mixture; and
(b) subjecting said reaction mixture to an amplification reaction.

25. The method of claim 24, wherein said reaction mixture further comprises a volume of between about 5 μL and about 200 μL.

26. The method of claim 24, wherein said reaction mixture further comprises a nucleoside triphosphate or a derivative thereof.

27. The method of claim 24, wherein said thermally stable enzyme is selected from the group consisting of polymerase, ligase, and combinations thereof.

28. The method of claim 24, wherein said reaction mixture further comprises a forward polynucleotide primer.

29. The method of claim 24, wherein said reaction mixture further comprises a reverse polynucleotide primer.

30. The method of claim 24, wherein said reaction mixture further comprises a probe.

31. The method of claim 24, wherein said reaction mixture further comprises a nucleic acid comprising said nucleic acid sequence.

32. The method of claim 24, wherein said reaction mixture further comprises HEPES.

33. The method of claim 24, wherein said reaction mixture further comprises an internal control.

34. The method of claim 24, wherein said reaction mixture further comprises a hot start methodology.

35. The method of claim 24, wherein said lyophilized bead has an average cross-section of between about 1 millimeter and about 4.5 millimeters.

36. A method for the amplification of a nucleic acid sequence, said method comprising:
(a) dissolving a lyophilized bead in a liquid, wherein said lyophilized bead comprises:
a forward polynucleotide primer;
a reverse polynucleotide primer; and
mannitol; and
wherein said lyophilized bead has a weight percentage of said mannitol of between about 53% and about 75% (w/w), thus forming a reaction mixture; and
(b) subjecting said reaction mixture to an amplification reaction.

37. The method of claim 36, wherein said reaction mixture further comprises a volume of between about 5 μL and about 200 μL.

38. The method of claim 36, wherein said reaction mixture further comprises a nucleoside triphosphate or a derivative thereof.

39. The method of claim 36, wherein said reaction mixture further comprises a probe.

40. The method of claim 36, wherein said reaction mixture further comprises a nucleic acid comprising said nucleic acid sequence.

41. The method of claim 36, wherein said reaction mixture further comprises HEPES.

42. The method of claim 36, wherein said reaction mixture further comprises a thermally stable enzyme.

43. The method of claim 36, wherein said reaction mixture further comprises an internal control.

44. The method of claim 36, wherein said lyophilized bead has an average cross-section of between about 1 millimeter and about 4.5 millimeters.

45. A lyophilized bead suitable for use in the amplification of a nucleic acid sequence, prepared by a process comprising:
(a) creating an aqueous solution, said aqueous solution comprising:
a thermally stable enzyme; and
mannitol;
wherein said solution has a concentration of said mannitol between about 0.38 M (moles of mannitol/liter of solution) and about 0.99 M (moles of mannitol/liter of solution);
(b) quick-freezing the product of (a); and
(c) freeze-drying the product of (b).

46. The lyophilized bead of claim 45, wherein the product of (c) has an average cross-section of between about 1 millimeter and about 4.5 millimeters.

47. The lyophilized bead of claim 45, wherein the product of (c) further comprises a nucleoside triphosphate or a derivative thereof.

48. The lyophilized bead of claim 45, wherein said thermally stable enzyme is selected from the group consisting of polymerase, ligase, and combinations thereof.

49. The lyophilized bead of claim 45, wherein the product of (c) further comprises a reverse transcriptase.

50. The lyophilized bead of claim 45, wherein the product of (c) further comprises a hot start methodology.

51. The lyophilized bead of claim 45, wherein the product of (c) further comprises HEPES.

52. The lyophilized bead of claim 45, wherein the product of (c) further comprises a probe.

53. The lyophilized bead of claim 45, wherein the product of (c) further comprises an internal control.

54. A lyophilized bead suitable for use in the amplification of a nucleic acid sequence, prepared by a process comprising:
(a) creating an aqueous solution, said aqueous solution comprising:
a forward polynucleotide primer;
a reverse polynucleotide primer; and
mannitol;
wherein said solution has a concentration of said mannitol between about 0.38 M (moles of mannitol/liter of solution) and about 0.99 M (moles of mannitol/liter of solution);
(b) quick-freezing the product of (a); and
(c) freeze-drying the product of (b).

55. The lyophilized bead of claim 54, wherein the product of (c) has an average cross-section of between about 1 millimeter and about 4.5 millimeters.

56. The lyophilized bead of claim 54, wherein the product of (c) further comprises a nucleoside triphosphate or a derivative thereof.

57. The lyophilized bead of claim 54, wherein the product of (c) further comprises HEPES.

58. The lyophilized bead of claim 54, wherein the product of (c) further comprises a probe.

59. The lyophilized bead of claim 54, wherein the product of (c) further comprises an internal control.

60. A lyophilized bead suitable for use in microanalytic systems comprising:
a moisture-sensitive reactant; and
mannitol;
wherein said lyophilized bead has a weight percentage of said mannitol of between about 53% and about 75% (w/w); and
wherein said lyophilized bead has an average cross-section of between about 1 millimeter and about 4.5 millimeters.

61. The lyophilized bead of claim 60, wherein said weight percentage is between about 62% and about 75% (w/w).

62. The lyophilized bead of claim 60, wherein said weight percentage is between about 68% and about 75% (w/w).
